(19) United States
(12) Patent Application Publication
(10) Pub. No.: US 2008/0172018 A1
(43) Pub. Date: Jul. 17, 2008

(54) PAD

(76) Inventor: Yuan-Cheng CHIEN, Kaohsiung Hsien (TW)

Correspondence Address: PATENTTM.US, P. O. BOX 82788, PORTLAND, OR, 97282-0788, US

(21) Appl. No.: 11/622,419

(22) Filed: Jan. 11, 2007

(51) Int. Cl. A61F 13/15

(52) U.S. Cl. 604/385.04; 604/385.01; 604/385.101

(57) ABSTRACT

A pad has an absorbing part. The absorbing part has a longitudinal groove and a guiding layer. The longitudinal groove is defined in middle of the pad. The guiding layer is mounted in the longitudinal groove and has a body and a line. The body is mounted in the longitudinal groove and has an upper end. The line connects with the upper end of the body. The line has two ends. The ends are held by user to pull up, so user can adjust the guiding layer to level at the crack between buttocks and absorb the menstrual blood from vagina immediately. Thus, user can sleep well, exercise freely and do not worry that the menstrual blood will flow out the pad to dirt the knickers or a bed sheet.

### BACKGROUND OF THE INVENTION

1. Field of Invention

The present invention relates to a pad, and more particularly to a pad that can be adjusted by users to guide and absorb menstrual blood immediately when menstrual blood flows out of vagina to prevent menstrual blood from flowing out of the pad.

2. Description of the Related Art

Ladies will worry about if pads are comfortable and can prevent menstrual blood from flowing out when ladies on their period. Thus, the pad has a front, a rear, two sides and an absorbing part. The rear of the pad may be elongated to prevent menstrual blood leaking out at night. Each side of the pad may have a wing to stick the pad securely on knickers, so ladies can exercise more freely. The absorbing part of the pad may have an all around flow guard to prevent menstrual blood from flowing out from the front, the rear and two sides of the pad. The pad may be scented to eliminate a peculiar smell.

However, an elongated pad for night can only prevent a part of menstrual blood from flowing out of the rear of the pad. Some menstrual blood will flow along a crack between buttocks and out of the pad when ladies sleep. Thus, menstrual blood will flow out of the pad to dirt knickers and a bed sheet and may remain in the crack between buttocks.

Someone invents a pad having a protrusion part formed on a central part of the absorbing part near the rear and protruding in the crack between buttocks to prevent the menstrual blood from flowing out of the pad and prevent menstrual blood from remaining in the crack of the buttocks. However, the protrusion part will be formed when the pad is unfolded, so the protrusion part is hard to be level at the crack between buttocks when a user wears the knickers. Moreover, when the user sleeps, she may turn aside and the menstrual blood may flow aside, so the protrusion part is useless. Thus, the pad with the protrusion part can not indeed prevent the menstrual blood from flowing out of the pad.

To overcome the shortcomings, the present invention provides a pad to mitigate or obviate the aforementioned.

### SUMMARY OF THE INVENTION

The primary objective of the present invention is to provide a pad that can be adjusted by users to guide and absorb menstrual blood immediately when menstrual blood flows out of vagina to prevent menstrual blood from flowing out of the pad.

To achieve the objective, the pad has an absorbing part. The absorbing part has a longitudinal groove and a guiding layer. The longitudinal groove is defined in middle of the pad. The guiding layer is mounted in the longitudinal groove and has a body and a line. The body is mounted in the longitudinal groove and has an upper end. The line connects with the upper end of the body. The line has two ends. The ends are held by user to pull up, so user can adjust the guiding layer to level at the crack between buttocks and absorb the menstrual blood from vagina immediately. Thus, user can sleep well, exercise freely and do not worry that the menstrual blood will flow out the pad to dirt the knickers or a bed sheet.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a pad in accordance with the present invention;

FIG. 2 is a perspective view in partial section of the pad in FIG. 1;

FIG. 3 is an operational perspective view of the pad in FIG. 1 with a first variant of a guiding layer being pulled up;

FIG. 4 is an enlarged cross sectional perspective view of the pad in FIG. 3 with the guiding layer being a piece of absorbing material being pulled up;

FIG. 5 is a side view of the pad in accordance with the present invention with a second variant of the guiding layer having a pharmaceutical part;

FIG. 6 is a side view of the pad in accordance with the present invention with a third variant of the guiding layer having multiple holes;

FIG. 7 is a side view of the pad in accordance with the present invention with a fourth variant of the guiding layer having a cotton line connecting with multiple cotton threads;

FIG. 8 is a side view of the pad in accordance with the present invention with a fifth variant of the guiding layer being a piece of foldable absorbing material;

FIG. 9 is a cross sectional end view of a second embodiment of the pad before a guiding layer be pulled up; and

FIG. 10 is an operational cross sectional end view of the pad in FIG. 9 with the guiding layer being pulled up.

### DETAILED DESCRIPTION OF THE INVENTION

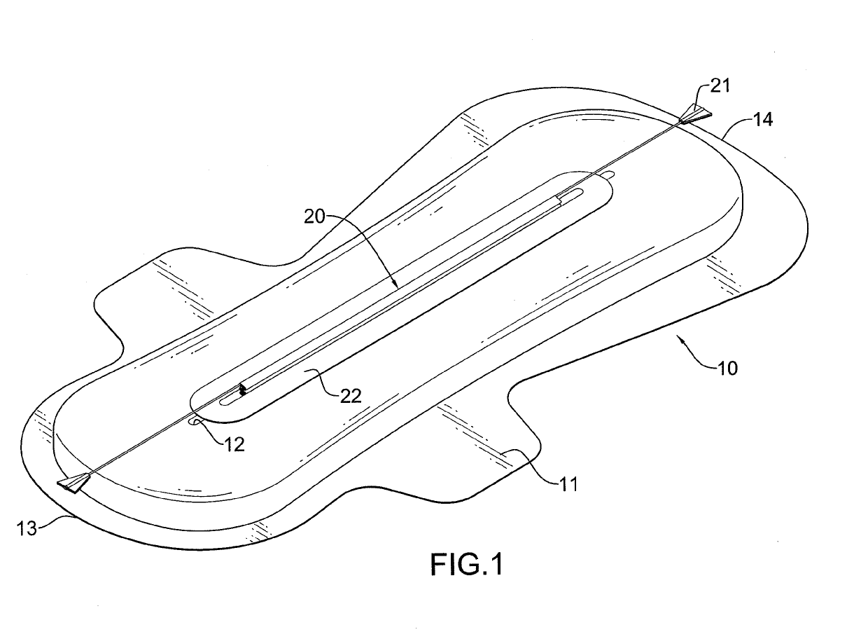

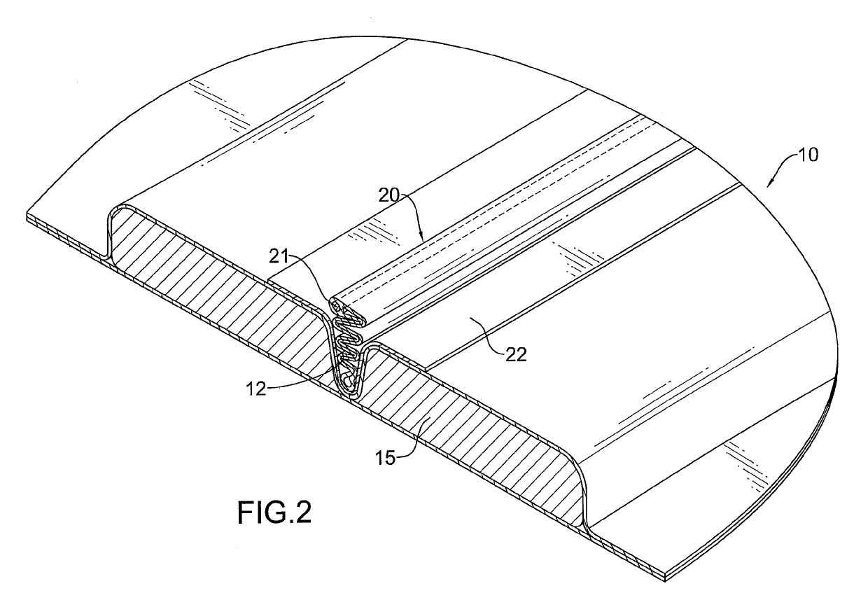

With reference to FIGS. 1 and 2, a pad (10) in accordance with the present invention has a lower surface, an upper surface, a front (13), a rear (14), two sides and an absorbing part.

The lower surface contacts a user's knickers and has adhesives to allow the pad (10) to stick on the knickers securely.

The upper surface is made of a nonwoven material to make the user comfortable.

The rear (14) of the pad (10) may be broadened or elongated.

Each side may have a wing (11). The wing (11) has a lower surface. The lower surface of the wing (11) has adhesives to stick on the knickers to allow the pad (10) to stick on the knickers securely.

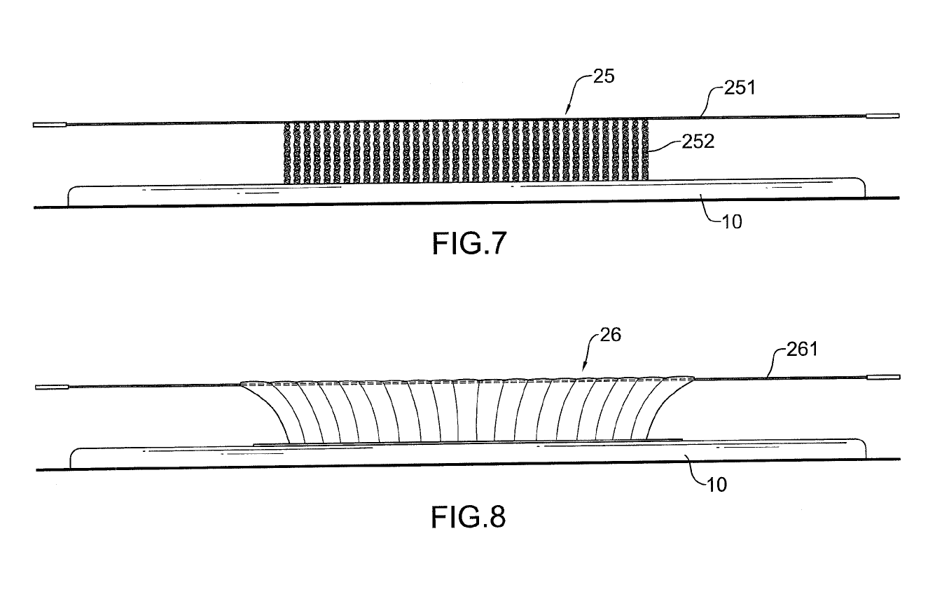

With further reference to FIGS. 7, 8, 9 and 10, the absorbing part has an inner space (15), a longitudinal groove (12, 32), a guiding layer (20, 25, 26, 30) and a barrier (22, 33).

The inner space (15) has multiple absorbing layers to absorb menstrual blood.

The longitudinal groove (12, 32) is defined in middle of the pad (10) and has a bottom. The bottom of the longitudinal groove (12, 32) is formed close to the lower surface of the pad (10).

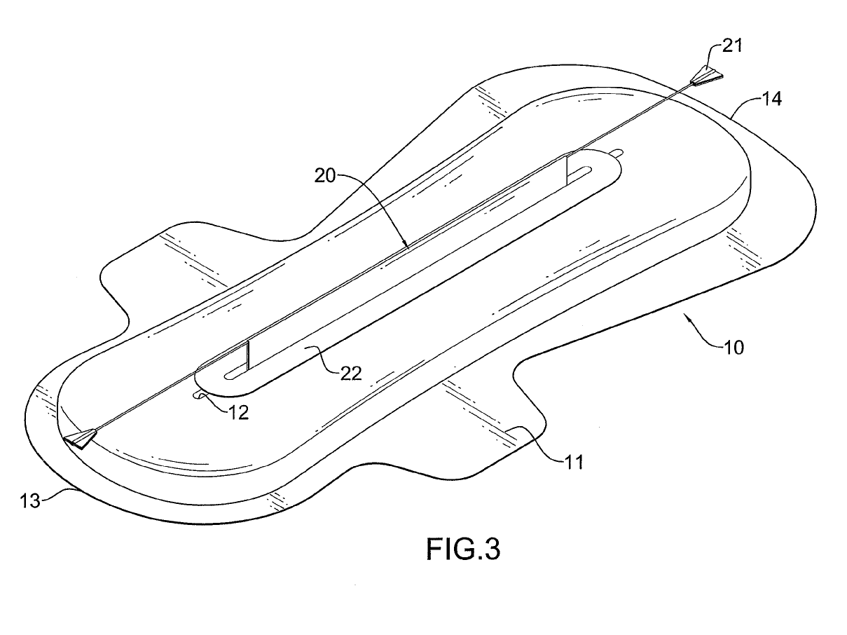

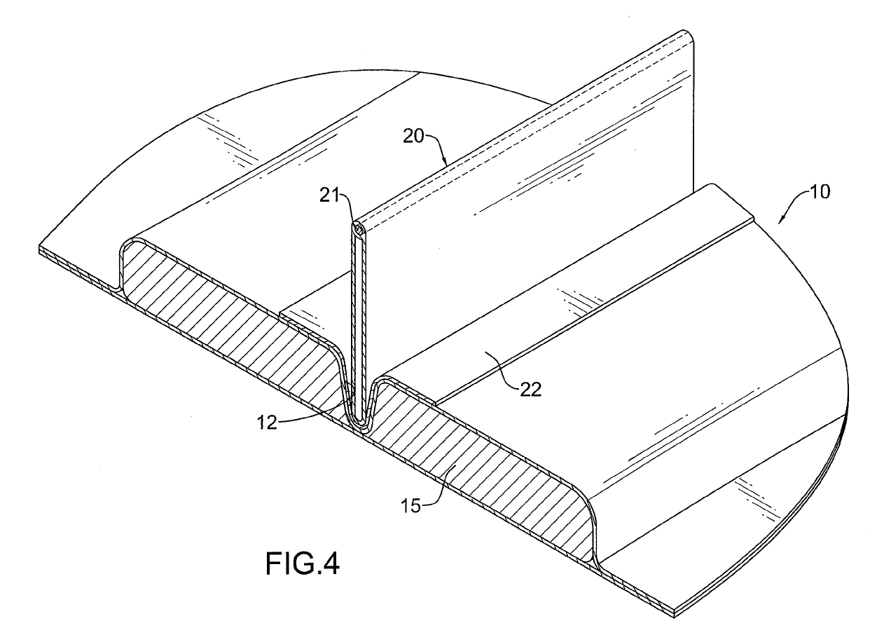

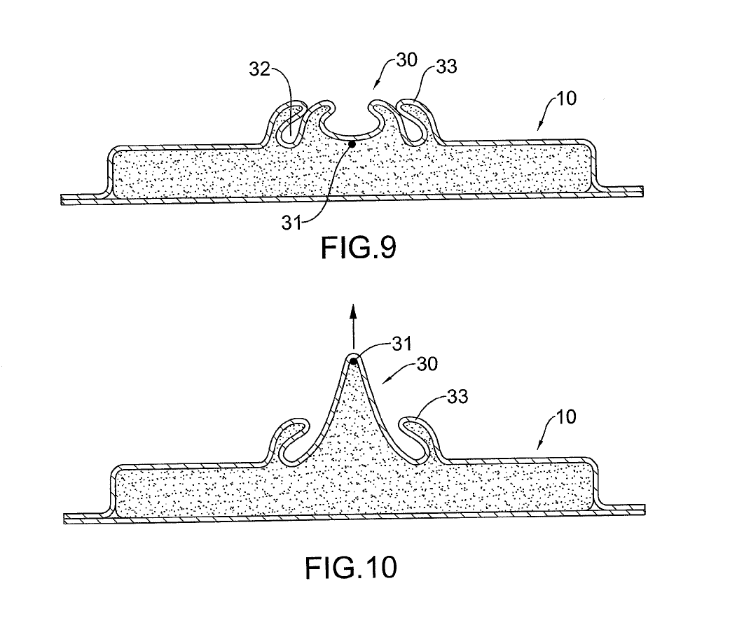

With further reference to FIGS. 3 and 4, the guiding layer (20, 25, 26 and 30) is mounted in the longitudinal groove (12, 32) and may connect with the bottom of the longitudinal groove (12, 32) in a first embodiment or may be formed integrally with the absorbing part in a second embodiment, as shown in FIGS. 9 and 10. The guiding layer (20, 25, 26 and 30) has a body and a line (21, 251, 261 and 31).

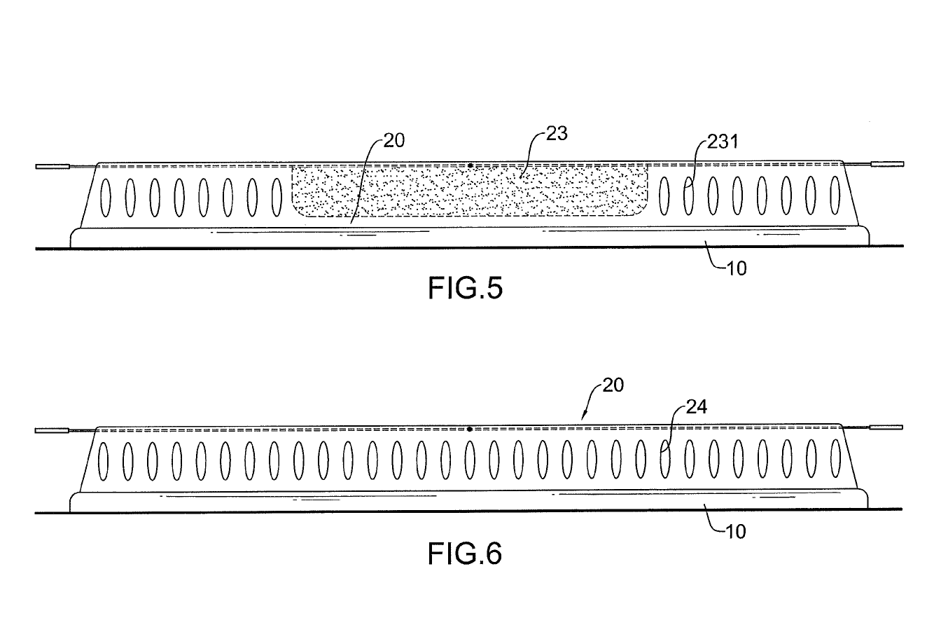

With reference to FIGS. 5 and 6, the body is mounted in the longitudinal groove (12, 32), may be a piece of foldable absorbing material (26) or may have multiple cotton threads (252) and has an upper end, a surface and may have multiple holes (231, 24). Each cotton thread (252) has an upper end. The upper end will contact a crack between buttocks and vagina to absorb the menstrual blood from vagina immediately. The surface may have a pharmaceutical part (23). The pharmaceutical part (23) has medicine. The medicine is adhered to the surface by coating or immersing to cure and prevent a bacterial infection. The holes (231, 24) are formed in the body to ventilate and make user to feel comfortable.

The line (21, 251, 261 and 31) connects with the upper end of the body and may be a cotton line (251) to connect with the upper ends of the cotton threads (252). The line (21, 251, 261 and 31) has two ends. The ends are held by user to pull up and the piece of absorbing material (26) will be unfolded and expanded, so user can adjust the guiding layer (20, 25, 26 and 30) to level at the crack between buttocks and absorb the menstrual blood from vagina immediately.

The barrier (22, 33) is mounted around the body of the guiding layer (20, 25, 26 and 30), protrudes from the upper surface of the pad (10), allows the menstrual blood to flow into the inner space of the pad (10) and prevents the menstrual blood from flowing out of the pad (10) from the front, the rear and the sides. The barrier (22) may be formed between the longitudinal groove (12) and the body of the guiding layer (20, 25 and 26).

The users can pull up the body of the guiding layer (20, 25, 26 and 30) by the line (21, 251, 261 and 31) and adjust the guiding layer (20, 25, 26 and 30) to level at the crack between buttocks and absorb the menstrual blood from vagina immediately. Thus, user can sleep well, exercise freely and do not that the menstrual blood will flow out the pad (10) to dirt the knickers or a bed sheet.

Even though numerous characteristics and advantages of the present invention have been set forth in the foregoing description, together with details of the structure and function of the invention, the disclosure is illustrative only. Changes may be made in detail, especially in matters of shape, size and arrangement of parts within the principles of the invention to the full extent indicated by the broad general meaning of the terms in which the appended claims are expressed.

What is claimed is:

1. A pad comprising:
an absorbing part having
a longitudinal groove being defined in middle of the pad;
a guiding layer mounted in the longitudinal groove and having
a body mounted in the longitudinal groove and having an upper end; and
a line connecting with the upper end of the body.

2. The pad as claimed in claim 1 further having
an upper surface;
a barrier mounted around the body of the guiding layer and protruding from the upper surface of the pad.

3. The pad as claimed in claim 1, wherein the body of the guiding layer has a surface having a pharmaceutical part comprising medicine adhered to the surface.

4. The pad as claimed in claim 1, wherein the body of the guiding layer has multiple holes formed in the body.

5. The pad as claimed in claim 1, wherein:
the body has multiple cotton threads and each cotton thread has an upper end; and
the line is a cotton line connecting with the upper ends of the cotton threads.

6. The pad as claimed in claim 1, wherein the body of the guiding layer is a piece of foldable absorbing material.

7. The pad as claimed in claim 1 further having two sides and each side having a wing having a lower surface comprising adhesives.

8. The pad as claimed in claim 1, wherein the longitudinal groove is formed integrally with the absorbing part.

9. The pad as claimed in claim 2, wherein the upper surface is made of a nonwoven material.

10. The pad as claimed in claim 7 further having a lower surface, wherein the longitudinal groove has a bottom formed close to the lower surface of the pad and the guiding layer connects with the bottom of the longitudinal groove.
